(19) United States
(12) Patent Application Publication
(10) Pub. No.: US 2025/0037408 A1
(43) Pub. Date: Jan. 30, 2025

(54) System and Method for Monitoring Critical Pharmaceutical Operations

(71) Applicant: Fresenius Kabi Austria GmbH, Graz (AT)

(72) Inventors: Christoph Köth, Graz (AT); Martin Kleinhenn, Gratkorn (AT); Philipp Kainz, Graz (AT); Andrea Maffeis, St. Josef (AT); Michael Mayrhofer-Reinhartshuber, Thalgau (AT); Thomas Ebner, Saalfelden (AT); Christina Egger, Graz (AT)

(21) Appl. No.: 18/716,826

(22) PCT Filed: Dec. 5, 2022
(86) PCT No.: PCT/EP2022/084391

§ 371 Date: Jun. 5, 2024

(30) Foreign Application Priority Data
Dec. 6, 2021 (EP) 21212628.8

(51) Int. Cl. G06V 10/25; G06V 10/764; G06V 20/40; G16H 40/20

(52) U.S. Cl. CPC G06V 10/25; G06V 10/764; G06V 20/44; G16H 40/20

(57) ABSTRACT

A system (1) for monitoring critical pharmaceutical operations comprises an enclosure (10) defining an interior space (100), at least one camera (11) installed so as to record image frames (F) of the interior space (100), and a controller (12), wherein the controller (12) is configured to: receive the image frames (F) recorded by the at least one camera (11), analyse the image frames (F) to detect an event captured by one or more of the image frames (F), using a first model perform a classification of an intervention captured by the one or more of the image frames (F) using a second model (ML 2), the second model (ML 2) being trained with image frames of interventions assigned to at least two different classes, and provide a notification (N) indicating one of the at least two different classes based on the classification.

The invention relates to a system and a method for monitoring critical pharmaceutical operations.

Many pharmaceutical operations, for example in an aseptic pharma production, have to be performed in a sterile environment typically provided by an isolator or similar system providing, e.g., clean room class A. Especially filling operations are often critical. All interventions, even when performed with glove protection, can negatively affect product sterility and are thus typically closely monitored, documented and analysed for a potential impact.

It is known from practice to use light barriers as safety precautions to automatically detect interventions. However, using light barriers, it is not possible to distinguish between different classes of interventions, e.g., between critical interventions and uncritical interventions.

EP 3 815 856 A1 describes an arrangement for monitoring a state and course of movement in an aseptic work chamber, comprising a tracking system with cameras. Therein, an exclusion area and a warning area are defined in the work chamber. A signal unit notifies a user of a motion in the warning area and alerts the user in case of a motion in the exclusion area. Such arrangements, however, generate many false alarms and require a very precise definition of the respective areas.

It is an object of the instant invention to improve the monitoring of critical pharmaceutical operations.

This object is achieved by means of a system comprising the features of claim 1.

Accordingly, a system for monitoring critical pharmaceutical operations (e.g., in an aseptic interior space), comprises an enclosure defining an interior space, at least one camera installed so as to record image frames of the interior space, and a controller. Therein, the controller is configured to: receive the image frames recorded by the at least one camera, analyse the image frames using a first model to detect an event, captured by one or more of the image frames. In case an event has been detected, perform a classification of an intervention captured by one or more of the image frames using a second model, the second model being trained with image frames of interventions assigned to at least two different classes, and provide a notification indicating one of the at least two different classes based on the classification.

For example, the second model classifies the interventions into critical interventions and non-critical interventions.

According to one embodiment of the invention, the first model is trained with image frames (training data) related to events, meaning with image frames showing events and image frames showing no events, respectively image data where no event happens, and the second model is trained with image frames (training data) of interventions (critical interventions and non-critical interventions).

For example, the first model is trained using training image frames with positive and negative classifications (i.e., results in a binary classifier). In other words, the first model is trained with images frames assigned to at least two different classes. An event may be detected if at least one frame (or, alternatively, at least another threshold number, e.g., 2, 3 or 4 of consecutive frames) are classified as showing an event are detected.

In other words, within the first model the image frames are classified as showing an event or not.

The training data may have been classified manually or using other reliable methods. Another set of pre-classified image frames may be used as test data set to test the performance of the first model and/or the second model.

This is based on the finding that a precise automatic classification of events and/or interventions can be made using the models trained with image frames showing events and showing interventions, wherein it is additionally not necessary to define exclusion areas with high precision. The models may be machine-learned models. The models may comprise Artificial Intelligence and Deep Learning.

An event may be the presence, respectively the beginning of a certain state and/or action, such as an intervention. An event may also be the ending of a certain state and/or action, such as an intervention.

Examples for events are: one or more cameras is/are obscured, a vial in the monitored area drops, a robotic arm approaches or moves into a pre-defined region, a door of the enclosure is opened, a glove port is in use, a glove port is no longer in use.

The critical pharmaceutical operations preferably comprise the production of medicine or medical nutrition or the like. In one embodiment the enclosure is equipped with instruments to perform the production of medicine or medical nutrition or the like.

In one embodiment the interior space is an aseptic interior space. The encloser is for example a clean room class A, a glove box, an isolator/RABS or the like.

In one embodiment the critical pharmaceutical operations comprise a pharmaceutical filling process, preferably an aseptic pharmaceutical filling process. To name few examples, an intervention may be a part of media filling processes, adjusting filling needles or a change of sedimentation disks, or even a person entering a clean room not wearing clothes suitable for the clean room classification.

The controller may be configured to detect an event using the first model by an analysis of at least one pre-defined first region of the respective image frames. Furthermore, the controller may be configured to classify a following intervention, using the second model, by an analysis of at least one pre-defined second region of the respective image frames. By this, the classification precision may be increased. Further, the necessary computing power may be decreased. Notably, such region or regions can be defined much coarser than strict exclusion areas described further above, because not a motion in the pre-defined region per se classifies an intervention, but the detection and classification of the intervention can be performed within this region. Preferably the pre-defined regions are also used within the training data.

The pre-defined first and second region may be of the same size and at the same position.

Alternatively, the pre-defined second region is smaller or larger than the pre-defined first region.

In general, these regions are independent in their size and position. One can say that especially the pre-defined second regions are chosen and placed such that they capture the regions an intervention is supposed to be detected and classified. For example, if an event has been detected, indicating the start of a filling needle adjustment (i.e. the intervention), the pre-defined second region(s) is (are) placed such that they depict the filling needles and vials which are supposed to be filled to capture the critical action of hovering over open vials or touching the needles for adjustment.

According to one embodiment more than one pre-defined first region of interest is analysed using the first model. Thus, more than one event may be detected simultaneously.

In addition, more than one pre-defined second region may be analysed using the second model. Thus, more than one intervention may be detected and classified simultaneously.

In one embodiment the viewing angle of the at least one camera, for example a wide-angle camera is fixed relative to the enclosure.

According to one further embodiment or in addition, multiple cameras and multiple pre-defined first regions and multiple pre-defined second regions from the different cameras are used for the classification, for example two different pre-defined first regions and two pre-defined second regions from two different cameras are used. This allows to detect and classify events and interventions using multiple viewing angles.

For example, at a fixed position in the image frames a pre-defined first region is defined. The pre-defined first region may for example be boxed shaped but could alternatively have another shape. The at least one pre-defined first region in the image frames is used by the controller using the first model to detect the start of an event. At a further fixed position in the image frames, at least one pre-defined second region is defined. The pre-defined second region may for example be boxed shaped but could alternatively have another shape. The pre-defined second region of the image frames is used by the controller using the second model, to classify an intervention. Image frames may be pre-processed.

The controller may be further configured to compute a difference image between a current frame and a reference frame. The difference image may be determined by subtracting the reference frame from the current frame (or vice versa). This allows to reduce the amount of information present in the current frame to the information relevant for the classification of the intervention. The reference frame may be the last image frame taken before an event has been detected.

Hence, the controller may be configured to perform the classification using the difference image. Using the second Model a classification is performed based on the difference image (D). Thereby, the accuracy of the classification can be increased.

For example, the reference frame is the last image frame before the event. By this, earlier changes inside the enclosure, e.g., earlier movements of items inside the enclosure, that are not related to the current event, can be ignored by using the last image frame before the event to compute the difference image. This allows a further increase of the classification accuracy.

In other words, the first model classifies every single frame according to whether it belongs to an event or not. For classification with the first model no reference frame(s) and/or difference images are used. Herein the first model may also be referred to as an event-detection model.

An intervention may be defined with a start and an end of an event. This means, if a predefined minimum number of frames is classified as belonging to an event, the intervention starts. The first frame of this group of frames taken within an event is defined as the start frame. The last frame taken before this group becomes the reference frame. In other words, the last frame take before an event has been detected is taken as a reference frame. On the other hand, if a predefined minimum number of frames is classified as not belonging to an event the intervention ends. The last frame belonging to the intervention is the one before this group. The first model may be trained with respective frames to detect the start and the end of an event. For example, the start of an event may be the beginning of a glove interaction and the end of the event is the following absence of a glove interaction. Another example might be an opening of a door (and entrance of a person) as start of an event and the consecutive re-opening of the door (and the person leaving). The actual event trigger can be learned from annotated data. If a user marks a certain period as an event, the first model tries to learn that frames in this period belong to an event and that the others do not.

Once the beginning of an event is detected a classification of an intervention within the event is performed using a second model. The second model classifies each frame of the intervention.

Preferably, an intervention is classified as critical if at least one frame is classified as critical.

Further, the controller may be configured to compute image features. The classification may be based on such image features.

Preferably the controller may be configured to compute the image features from the respective pre-defined first and second regions only in order to reduce the necessary computing power. Additionally, the pre-defined first and/or second regions may be cropped in size.

For example, the controller is configured to compute a histogram of oriented gradients, HOG. A HOG can be used to represent low-level image features by a set of numbers that can more robustly be interpreted by a model.

For instance, within the first model the pre-defined first region of the current frame is used to compute the HOG, in the second model pre-defined second region of the difference image is used.

According to one embodiment each frame is classified as critical or as non-critical. For this classification the second model uses a difference image computed between a current frame and the reference frame. For example, a histogram of oriented gradients (HOG) is computed. The controller assigns, via the second model, a score (a number between 0 and 1) to each frame. In case the score is above a predefined threshold (e.g. 0.5) the frame is classified as critical.

Optionally, the second model may be adapted to assign each of a plurality of HOG features a value. These values may indicate the contribution of the respective HOG feature to the classification of the frame. Further these values may be displayed. Thus, a user may directly gain insights on the reasons of the classification by the second model. By this, one major challenge in the use of common black box models may be alleviated, namely, the explanation of the reasons of the decision made by the model. Thus, the described system may provide an XAI (eXplainable Artificial Intelligence) component.

The controller can be further configured to generate a graphical representation of the HOG for presentation on a display device. This allows a user of the system to directly gain insights on the reasons of the classification by the second model.

Together, the image frames may form a video stream. The controller may be adapted to perform the classification in real-time with respect to the video stream. This allows to classify an intervention without or with negligible delay. Herein, e.g., the processing of an image frame does not take more time than the delay between two consecutive image frames in accordance with the frame rate of the image frames.

Optionally, the controller is further configured to detect the time and/or location within the interior space of the detected event.

For example, the at least two different classes indicate whether or not the detected intervention is critical or non-critical for a process performed within the interior space. As such, the system may detect critical interventions and the provided notification can inform the user about the criticality.

The system may for example comprises an alarm device, capable of giving an alarm, for example a visual or acoustic alarm signal in case an intervention has been classified as critical. The notification provided by the controller is for example displayed on a display device as alarm device.

The alarm signal may be connected to the controller via wireless or wire connection.

The controller may comprise a processor and memory. The memory stores executable code and the first and second model.

According to one embodiment, the enclosure, for example a glove box comprises one or more glove ports. Glove ports allow for interventions in the interior space of the enclosure maintaining an isolation of the enclosure.

The controller may be further configured to determine whether or not a respective glove of each of the one or more glove ports is arranged inside or outside of a wall of the enclosure. This may be performed by a means of a classification.

According to an embodiment, the pre-defined first region of the respective image frames depicts at least one of the one or more glove ports. Preferably image frames and therein pre-defined first regions of all glove ports from which critical interventions may be performed, are analysed. In other words, all glove ports are monitored from which critical areas within the encloser may be reached.

An event is then detected by the controller using the first model when a respective glove is detected as being inside the enclosure. The intervention comprises an action performed using at least one of the one or more glove ports. The system can thus detect and classify interventions performed at the glove ports. The event, particularly the intervention, is defined by a time period during which a motion takes place, for example a glove insertion, particularly a potentially critical action. An intervention is for example defined by the time period between a detected start of an event (e.g., glove interaction) and the detected end of the event (glove interaction).

According to an aspect, a method for monitoring critical pharmaceutical operations is provided. The method comprises receiving, by a controller, image frames recorded by at least one camera, the at least one camera being installed so as to record the image frames of an interior space defined by an enclosure, analysing, by the controller, the image frames using a first model to detect an event captured by one or more of the image frames, performing, by the controller, a classification of an intervention captured by the one or more of the image frames using a second model, and providing, by the controller, a notification indicating one of the at least two different classes based on the classification.

In one embodiment the first model is trained with image frames of events and the second model is trained with image frames of interventions.

Single steps of the method according to an embodiment will be described in more detail in the following. In a first step the controller receives image frames recorded by the at least one camera, the at least one camera being installed so as to record the image frames of the interior space defined by the enclosure. The processing of the image frames is performed in a two-stage computer vision algorithm, comprising second and third steps. In a second step the controller analyses the image frames to detect an event captured in one or more of the image frames.

To detect the event, a pre-defined first region is analysed by a trained machine learning first model for event detection (event-detection model). The trained event-detection model is stored in a memory. For this analysis, the controller calculates a histogram of oriented gradients, HOG, for the respective pre-defined first regions, which is provided to the event-detection model (first model) as input. The event-detection model determines a classification result which is either positive (event detected) or negative (no event detected). The event-detection model is trained using training image frames (in particular, with the respective HOGs) with positive and negative classifications (i.e., results in a binary classifier).

As an example, an intervention may be defined as being imminent if one of the gloves is inside the enclosure. Correspondingly, when no glove is inside the enclosure, the respective image frame may be defined as not depicting an intervention. Optionally, different types of events may be detected. For example, a Random Forest algorithm is used as the event-detection model. Accordingly, in one embodiment, a Random Forest is used as the first model ML1 being an event-detection model. Optionally, an event may be detected if at least one frame (or, alternatively, at least another threshold number, e.g., 2, 3 or 4 of consecutive frames) are classified as showing an event are detected.

In a third step the controller performs a classification of the detected intervention captured by the one or more of the image frames classified as showing an event, using a second model as classification model. As soon as an event is detected starting with a given image frame, the last image frame before that has not been classified as showing an event is defined as a reference frame. In this step, a current frame currently being classified, and the reference frame RF are used to compute a difference image. This difference image is used for the analysis. Here, the difference image is used to compute HOG features which are then input to the classification model. Once a single image frame is detected as critical, the whole intervention is considered critical. Critical sequences also contain non-critical image frames, typically in the beginning and at the end, and at least one critical image frame. The end of an event is determined when a threshold number (e.g., 1, 2, 3 or 4) of consecutive image frames are classified as not showing an event. Each event has a corresponding reference frame. That is, for every newly detected event, a respective reference frame is determined.

In the present example, the third step is only performed for image frames after an event is detected in the second step The second model is trained with image frames of interventions (in general: actions) assigned to at least two different classes, here: critical or non-critical. The second model is trained using training image frames (in particular, with the respective HOGs) from critical and non-critical interventions (i.e., yields another binary classifier). In the present example, another binary Random Forest algorithm is used as the second model. For example, a critical image frame may be one where the glove touches a given surface or is too close to a given object. To name few examples, an intervention may be a part of media filling processes, adjusting filling needles or a change of sedimentation disks. Accordingly, in one embodiment, another Random Forest is used as the second model ML 2 for intervention classification.

In one embodiment SHapely Additive exPlanations (SHAP) are applied to visualize the HOG features in an image. This allows a user to gain insights into why the Random Forest classified image frames as critical or non-critical.

Optionally, additional parameters are used to calculate the probability that the intervention is critical, e.g., the duration of the intervention.

The second and third steps are performed for pre-defined first and pre-defined second regions individually. Thus, more than one event may be detected simultaneously and more than one intervention may be classified simultaneously. For example, one (e.g., non-critical) intervention at one glove port may be performed at the same time as another (e.g., critical) intervention at another glove port.

The training data may have been classified manually or using other reliable methods. Another set of pre-classified image frames may be used as test data set to test the performance of the event-detection model and/or the classification model.

In a fourth step the controller provides a notification indicating one of the at least two different classes based on the classification. Optionally, the system and method record all recognized interventions (more general: actions) and parameters thereof (e.g., date and time, duration, type of intervention etc.). Then the operator may be notified of upcoming required interventions. The record may be used for quality control and assurance and/or to trigger corrective actions depending on the recognized interventions. Accordingly, in one embodiment, the system is configured to record the interventions and parameters thereof, e.g., date, time, duration and/or type of intervention time. Preferably, the system is further configured to document the interventions. This documentation enables an analysis of interventions for a potential impact, for instance negatively affected product sterility. As an example, one or more already filled vials due to the detection of critical interventions could be rejected.

The method may use the system in accordance with any aspect or embodiment described herein. Regarding the advantages of the method reference is made to the description of the system above.

It is to be understood and obvious to those skilled in the art, that when it is said that image frames are processed or computed by the controller, this does not necessarily mean the whole image frames as they were recorded by the one or more camera are used for classification. The controller may first pre-process the image frames to make them suitable for the first and second model.

### EMBODIMENTS OF THE INVENTION

Embodiment 1: A system for monitoring critical pharmaceutical operations, the system comprising an enclosure defining an interior space, at least one camera installed so as to record image frames of the interior space, and a controller, wherein the controller is configured to:
receive the image frames recorded by the at least one camera,
analyse the image frames to detect an event captured by one or more of the image frames using a first model,
perform a classification of an intervention captured by one or more of the image frames using a second model, the second model being trained with image frames of interventions assigned to at least two different classes, and
provide a notification indicating one of the at least two different classes based on the classification.

Embodiment 2: The system according to embodiment 1, wherein the first model is trained with image frames of events and/or the second model is trained with image frames of interventions, preferably the first and the second model are machine-learned-models.

Embodiment 3: The system according to embodiment 1 or 2, wherein the controller is configured to detect the event by an analysis of a pre-defined first region of the respective image frames and/or in that the controller is configured to perform a classification of the intervention by an analysis of a pre-defined second region of the respective image frames.

Embodiment 4: The system according to any of the preceding embodiments, wherein the controller is further configured to compute a difference image between a current frame and a reference frame.

Embodiment 5: The system according to embodiment 4, wherein the controller is configured to compute a difference image between a current frame and a reference frame, using the second Model the classification is performed based on the difference image.

Embodiment 6: The system according to embodiments 4 or 5 wherein the reference frame is the last image frame before the event.

Embodiment 7: The system according to any of the embodiments 4 to 6, wherein the controller is further configured to compute image features using the difference image.

Embodiment 8: The system according to any of embodiments 4 to 7, wherein the controller is configured to compute a histogram of oriented gradients, HOG, using the difference image.

Embodiment 9: The system according to embodiment 8, wherein the trained second model is adapted to assign each of a plurality of HOG features a value indicating a contribution of the respective HOG feature to the classification of the intervention.

Embodiment 10: The system according to embodiment 8 or 9, wherein the controller is further configured to generate a graphical representation of the HOG for presentation on a display device.

Embodiment 11: The system according to any of the preceding embodiments, wherein the image frames form a video stream, wherein the controller is adapted to perform the classification in real-time with respect to the video stream.

Embodiment 12: The system according to any of the preceding embodiments, wherein the controller is further configured to detect the time and/or location within the interior space of the detected event.

Embodiment 13: The system according to any of the preceding embodiments, wherein the at least two different classes indicate whether or not the detected intervention is critical or non-critical for a process performed within the interior space.

Embodiment 14: The system according to any of the preceding embodiments, wherein the enclosure comprises one or more glove ports.

Embodiment 15: The system according to embodiment 14, wherein the controller is further configured to determine whether or not a respective glove of each of the one or more glove ports is arranged inside or outside of a wall of the enclosure.

Embodiment 16: The system according to embodiment 14 or 15, wherein the pre-defined region of the respective image frames depicts at least one of the one or more glove ports.

Embodiment 17: The system according to any of embodiments 14 to 16, wherein, the event is the start of an intervention, wherein, optionally, the intervention comprises an action performed using at least one of the one or more glove ports.

Embodiment 18: A method for monitoring critical pharmaceutical operations, comprising:
receiving, by a controller, image frames recorded by at least one camera, the at least one camera being installed so as to record the image frames of an interior space defined by an enclosure,
analysing, by the controller, the image frames using a first model to detect an event captured by one or more of the image frames,
performing, by the controller, a classification of an intervention captured by the one or more of the image frames using a second model, the second model being trained with image frames of interventions assigned to at least two different classes, and
providing, by the controller, a notification indicating one of the at least two different classes based on the classification.

The idea underlying the invention shall subsequently be described in more detail by referring to the embodiments shown in the figures. Herein:

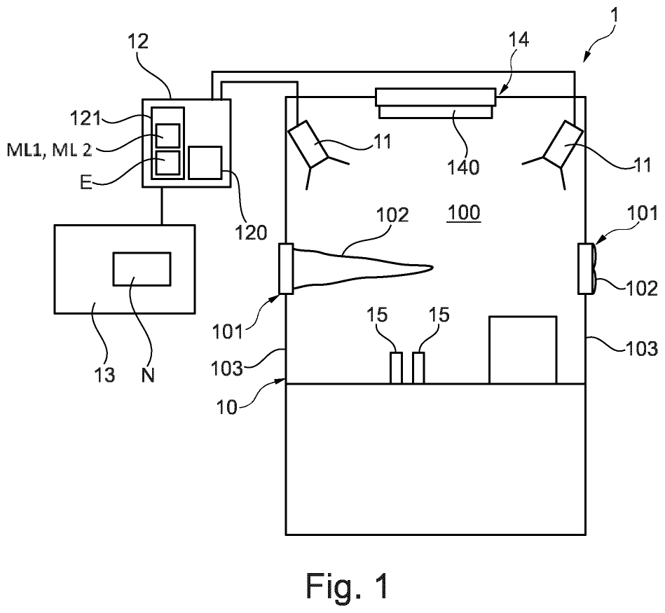

FIG. 1 shows a system for monitoring critical pharmaceutical operations in an aseptic interior space using two cameras and first and second models;

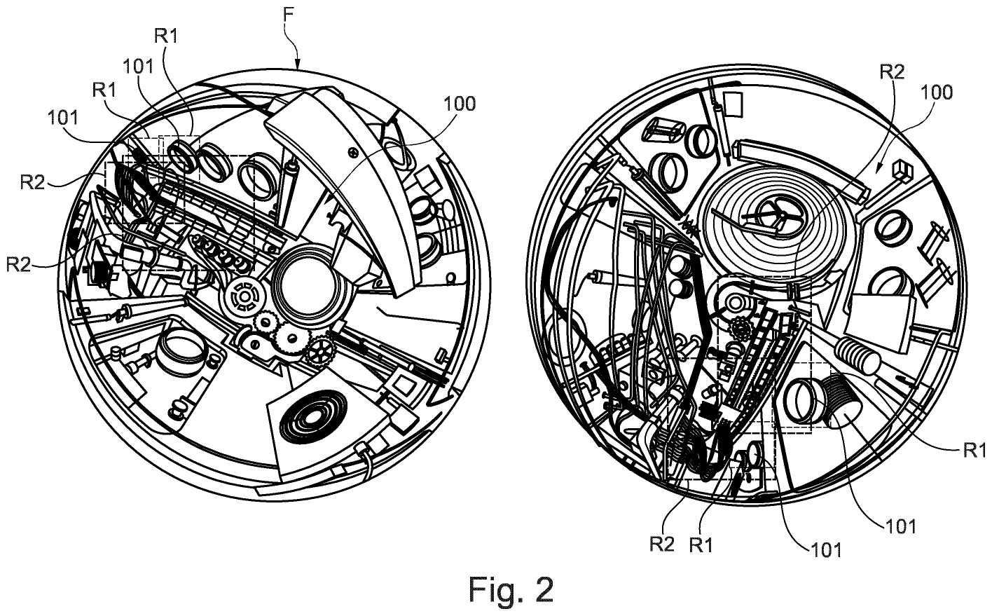

FIG. 2 shows an image frame assembled from images taken by the two cameras, showing the interior space;

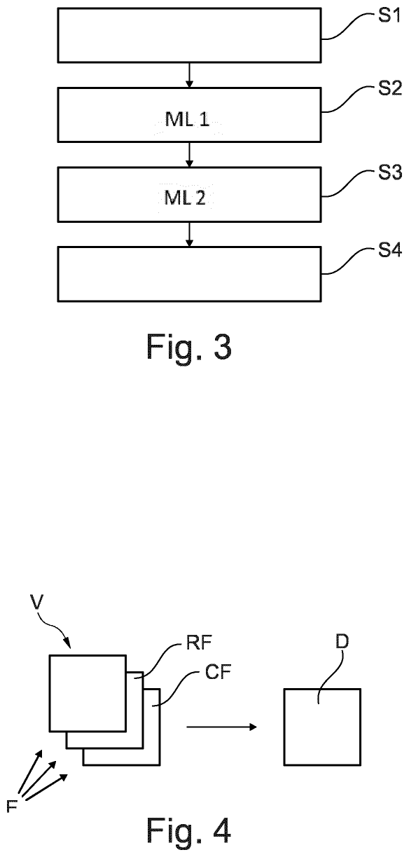

FIG. 3 shows a method for monitoring critical pharmaceutical operations in an aseptic interior space using two cameras and first and second models;

FIG. 4 shows a video stream comprising several image frames, and a difference image computed based on a reference frame and a current frame;

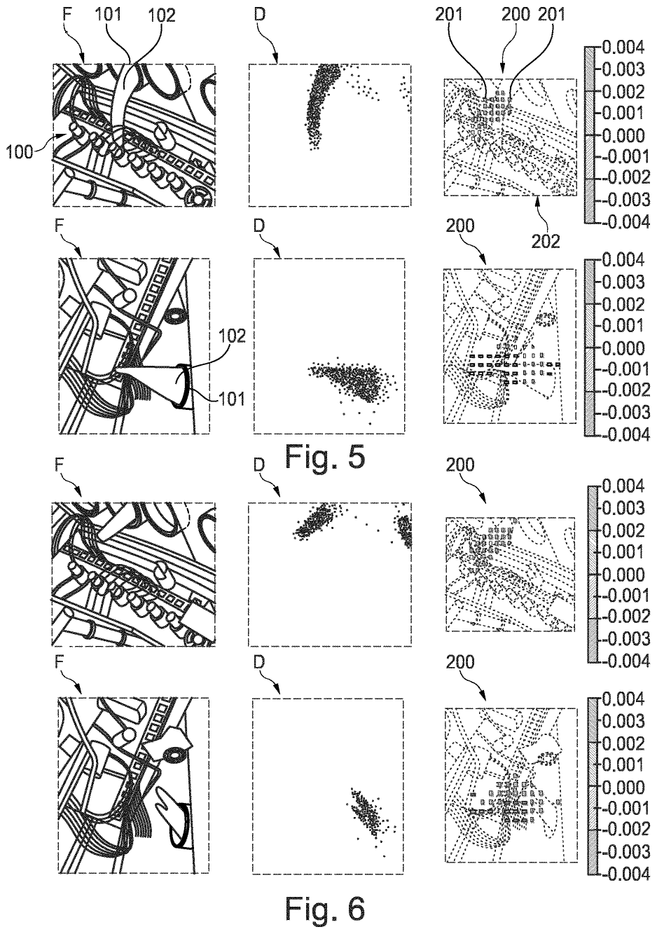

FIG. 5 shows parts of image frames of a critical intervention recorded by the two cameras, a respective difference image, respective difference images and histograms of oriented gradients; and

FIG. 6 shows parts of images of an uncritical intervention recorded by the two cameras, a respective difference image, respective difference images and histograms of oriented gradients.

Subsequently, a system and method for monitoring critical pharmaceutical operations shall be described in certain embodiments. The embodiments described herein shall not be construed as limiting for the scope of the invention.

FIG. 1 shows a system 1 for monitoring critical pharmaceutical operations in an aseptic interior space 100.

The system 1 comprises an enclosure 10 defining the interior space 100, generally one or more cameras 11, here two cameras 11, are installed so as to record image frames of the interior space 100. Here, the cameras are arranged at an upper area of the enclosure 10 (inside the interior apace 100) facing downwards.

The enclosure 10 comprises walls 103. The walls 103 delimit the interior space 100. The walls 103 isolate the interior space 100 from the surrounding environment.

Inside the interior space 100 various items are arranged, such as vials 15. The enclosure 10 is equipped with instruments to perform critical pharmaceutical operations, e.g., the production of medicine or medical nutrition or the like.

The system 1 further comprises glove ports 101. The enclosure 10 is a glove box. Each of the glove ports 101 is mounted in one of the walls 103 of the enclosure 10. The walls 103 may be glass panels. Each glove port 101 comprises a glove 102. An operator may insert a hand into one or more of the gloves 102. For illustrative purposes, one glove 102 (the left one in FIG. 1) is shown in a state inside the interior space 100, while the other glove 102 (the right one in FIG. 1) is shown in a state not inserted into the interior space 100. The glove ports 101 and the gloves 102 are within the field of view of each of the cameras 11 (generally, of at least one of the cameras 11).

The system 1 comprises a ventilation 14. The ventilation 14 comprises an air filter 140. The air filter 140 is adapted to filter air supplied to the enclosure. The air filter 140 is adapted to filter dust and germs from the air. The enclosure 10 of FIG. 1 is an isolator. An isolator is a type of clean air device that creates an almost complete separation between a product and production equipment, personnel, and surrounding environment. Operators who operate a production line can take actions inside isolators via the glove ports 101 in order to perform tasks required for the production process (required interventions, e.g., sedimentation disk changes) or to perform manipulations of objects/devices to maintain the production process (maintenance interventions, e.g., removing empty vials that fell off a conveyor). These interventions have to be documented and further measures have to be taken depending on the parameters (position, time, duration and/or class (e.g., critical or non-critical intervention)) of the interventions performed (e.g., rejecting one or more already filled vials due to the detection of critical interventions). Notably, however, aseptic filling is not limited to isolators.

Aseptic filling and other critical pharmaceutic operations can also be performed in specially designed clean rooms (class A with background cleanroom class B) or in RABS (restricted access barrier system) installations. Those impose a much higher risk to the product compared to isolator operations and interventions must be even closer monitored but are still widely used in pharma production.

Further, the system 1 comprises a controller 12 configured to receive the image frames recorded by the cameras 11, to analyse the image frames to detect an event captured by one or more of the image frames using a first model ML1. To perform a classification of an intervention captured by one or more of the image frames the controller uses a second model ML 2, the second model ML2 being trained with image frames of interventions assigned to at least two different classes, and to provide a notification N indicating one of the at least two different classes based on the classification.

The event may be an intervention, e.g., an intervention of at least one operator. For example, the intervention is an action performed inside the interior space. The intervention may be performed via one or more of the glove ports.

For example, for the at least two difference classes it is distinguished between critical and non-critical interventions. Critical interventions comprise at least one critical image frame. The single image frames during one intervention are assigned to critical frames and uncritical frames.

To detect and classify events and/or interventions within the interior space 100, the controller 12 is connected to the cameras 11 so as to receive a video stream of image frames from each of the cameras 11. The controller 12 comprises a processor 120 and a memory 121. The memory 121 stores executable code E and the first and second model. The notification N provided by the controller 12 is displayed on a display device 13.

FIG. 2 shows a combined image frame F comprising an image frame of each of the cameras 11. This allows a simplified processing, but it is worth noting that the image frames of both cameras 11 could also be processed independently in parallel.

The viewing angle of each of the cameras 11 is fixed relative to the enclosure 10. As an example, two of the glove ports 101 are monitored. It will be appreciated, however, that more than two, e.g., all glove ports 101 of the system 1 may be monitored.

At fixed positions in the image frame F, pre-defined first regions R1 at the monitored glove ports 101 are defined. Here, each of the pre-defined first regions R1 includes one of the glove ports 101. The pre-defined first regions R1 are box shaped but could alternatively have another shape. At further fixed positions in the image frame F, pre-defined second regions R2 at the monitored glove ports 101 are defined. Here, each of the pre-defined second regions R2 includes at least a part of one or more of the glove ports 101. The pre-defined second regions R2 are box shaped but could alternatively have another shape. For each monitored glove port 101 a respective pre-defined first region R1 and a respective pre-defined second region R2 may be defined. Each pre-defined second region R2 comprises a larger area than the corresponding pre-defined first region R1.

When executed by the processor 120, the executable code E stored in the memory 121 causes the processor 120 to perform the method of FIG. 3. In the method, the following steps are performed:

Step S1: Receiving, by the controller 12, image frames F recorded by the at least one camera 11, the at least one camera 11 being installed so as to record the image frames F of the interior space 100 defined by the enclosure 10. The processing of the image frames is performed in a two-stage computer vision algorithm, comprising steps S2 and S3.

Step S2: Analysing, by the controller 12, the image frames F to detect an event captured in one or more of the image frames F. To detect the event, the pre-defined first regions R1 (see FIG. 2) are analysed by a trained machine learning first model (ML 1) for event detection (event-detection model). The trained event-detection model is stored in the memory 121. For this analysis, the controller 12 calculates a histogram of oriented gradients, HOG, for the respective pre-defined first regions R1, which is provided to the event-detection model (first model) as input. The event-detection model determines a classification result which is either positive (event detected) or negative (no event detected). The event-detection model is trained using training image frames (in particular, with the respective HOGs) with positive and negative classifications (i.e., results in a binary classifier). As an example, an intervention may be defined as being imminent if one of the gloves 102 is inside the enclosure 10. Correspondingly, when no glove 102 is inside the enclosure 10, the respective image frame F may be defined as not depicting an intervention. Optionally, different types of events (particularly interventions) may be detected. In the present example, a Random Forest algorithm is used as the event-detection model. Optionally, an event may be detected if at least one frame (or, alternatively, at least another threshold number, e.g., 2, 3 or 4 of consecutive frames) are classified as showing an event are detected.

Step S3: Performing, by the controller 12, a classification of the detected intervention captured by the one or more of the image frames F classified as showing an event, using a second model ML 2 as classification model. As soon as an event is detected starting with a given image frame F, the last image frame F before that has not been classified as showing an event is defined as a reference frame RF. In step S3, a current frame CF currently being classified, and the reference frame RF are used to compute a difference image D, see FIG. 4. This difference image D is used for the analysis. Here, the difference image D is used to compute HOG features which are then input to the classification model ML 2. Once a single image frame F is detected as critical, the whole intervention is considered critical. Critical sequences also contain non-critical image frames F, typically in the beginning and at the end, and at least one critical image frame F. The end of an event is determined when a threshold number (e.g., 1, 2, 3 or 4) of consecutive image frames F are classified as not showing an event. Each event has a corresponding reference frame RF. That is, for every newly detected event, a respective reference frame RF is determined.

In the present example, step S3 is only performed for image frames F after an event is detected in step S2. The second model ML 2 is trained with image frames F of interventions (in general: actions) assigned to at least two different classes, here: critical or non-critical. The second model ML 2 is trained using training image frames (in particular, with the respective HOGs) from critical and non-critical interventions (i.e., yields another binary classifier). In the present example, another binary Random Forest algorithm is used as the second model ML 2. For example, a critical image frame may be one where the glove 102 touches a given surface or is too close to a given object. To name few examples, an intervention may be a part of media filling processes, adjusting filling needles or a change of sedimentation disks.

Optionally, additional parameters are used to calculate the probability that the intervention is critical, e.g., the duration of the intervention.

Steps S2 and S3 are performed for each glove port 101 individually. Thus, more than one event may be detected simultaneously. For example, one (e.g., non-critical) intervention at one glove port 101 may be performed at the same time as another (e.g., critical) intervention at another glove port 101.

The training data may have been classified manually or using other reliable methods. Another set of pre-classified image frames may be used as test data set to test the performance of the event-detection model and/or the classification model.

Step S4: Providing, by the controller 12, a notification N indicating one of the at least two different classes based on the classification. Optionally, the system 1 and method record all recognized interventions (more general: events) and parameters thereof (e.g., date and time, duration, type of intervention etc.). Then the operator may be notified of upcoming required interventions. The record may be used for quality control and assurance and/or to trigger corrective actions depending on the recognized interventions.

The method is performed in real-time (alternatively, post-hoc) on a video stream V (see FIG. 4) comprising a sequence of image frames F. The frame rate may be, e.g., between 5 and 20 frames per second, particularly 10 frames per second.

Turning now to FIGS. 5 and 6 the functionality of the classification will be described in more detail.

FIG. 5 shows on the left image frames F of the two cameras 11 showing a critical intervention. In the middle, corresponding difference images D are shown. On the right, graphical representations 202 comprising the corresponding HOGs 200 are shown. Each HOG 200 comprises a plurality of HOG features 201. Each HOG feature 201 is assigned, by means of the second classification model ML 2, a value which corresponds to its contribution to the model's decision.

FIG. 6 shows the same as FIG. 5, just for a non-critical intervention.

To allow a user to gain insights into why the Random Forest classified image frames F as critical or non-critical, the graphical representations 202 are displayed, e.g., on display device 13. Here, the HOG features 201 may be overlaid the respective image frame F (optionally shaded). More specifically, SHapely Additive exPlanations (SHAP) are applied to visualize the HOG features 201 in an image. FIGS. 5 and 6 show positive SHAP values (towards green. here hatched illustrated, contribute to a non-critical decision) and negative SHAP values (towards red, here hatched illustrated, contribute to a critical decision).

Thus, while usually an AI component in an analysis must be regarded as a black box, here it is possible to directly visualize the data which is the basis for the decision of the second model ML 2. This allows more reliable results and simplified certification in many fields of application.

The basic idea of HOG is that based on the gradients (intensity differences of neighbouring pixels) a robust colour and size independent objective description of the image content is obtained. The entire image section used for classification (second regions R2) is scaled to a fixed size and divided into 8×8 pixel cells in which a histogram is formed over the 9 main directions (0-360°). That is, each cell is described by a 9-bin histogram. Then, these features are normalized, and the histograms are lined up. This then results in a feature vector, where each number in the vector is called a feature.

While above the event has been described as an intervention using a glove port 101, it will be appreciated that the same algorithm may be applied for other kinds of events. Indeed, the system 1 does not necessarily have to comprise glove ports 101 at all.

Notably, in addition to the one or more cameras 11 other sensor types may be used to provide input to the analysis described above, e.g., LiDAR sensors.

The idea of the invention is not limited to the embodiments described above but may be implemented in a different fashion.

### LIST OF REFERENCE NUMERALS

1 System
10 Enclosure
100 Interior space
101 Glove port
102 Glove
103 Wall
11 Camera
12 Controller
120 Processor
121 Memory
13 Display device
14 Ventilation
140 Air filter
15 Vial
200 Histogram of oriented gradients, HOG
201 HOG feature
202 Graphical representation
CF Current frame
D Difference image
E Executable code
F Image frame
M Model
N Notification
R1, R2 Region
RF Reference frame
V Video stream

1. A system for monitoring critical pharmaceutical operations, the system comprising an enclosure defining an interior space, at least one camera installed so as to record image frames of the interior space, and a controller, wherein the controller is configured to:
receive the image frames recorded by the at least one camera,
analyse analyze the image frames to detect an event captured by one or more of the image frames using a first model,
perform a classification of an intervention captured by one or more of the image frames using a second model, the second model being trained with image frames of interventions assigned to at least two different classes, and
provide a notification indicating one of the at least two different classes based on the classification.

2. The system according to claim 1, wherein the first model is trained with image frames related to events and/or the second model is trained with image frames of interventions, preferably the first and the second model are machine-learned-models.

3. The system according to claim 1, wherein the controller is configured to detect the event by an analysis of at least one pre-defined first region of the respective image frames and/or in that the controller is configured to perform a classification of the intervention by an analysis of at least one pre-defined second region of the respective image frames.

4. The system according to claim 3, wherein the controller is configured to compute a difference image between a current frame and a reference frame, using the second model the classification is performed based on the difference image.

5. The system according to claim 4, wherein the reference frame is a last image frame before the event.

6. The system according to claim 4, wherein the controller is further configured to compute image features using the difference image.

7. The system according to claim 4, wherein the controller is configured to compute a histogram of oriented gradients, HOG, using the difference image.

8. The system according to claim 7, wherein the second model is adapted to assign each of a plurality of HOG features a value indicating a contribution of the respective HOG feature to the classification of the intervention.

9. The system according to claim 7, wherein the controller is further configured to generate a graphical representation of the HOG for presentation on a display device.

10. The system according to claim 1, wherein the image frames form a video stream, wherein the controller is adapted to perform the classification in real-time with respect to the video stream.

11. The system according to claim 7, wherein a Random Forest is used as the first model for event detection.

12. The system according to claim 7, wherein another Random Forest is used as the second model for intervention classification.

13. The system according to claim 8, wherein SHapely Additive exPlanations are applied to visualize the plurality of HOG features in an image.

14. The system according to claim 1, wherein the at least two different classes indicate whether or not the detected intervention is critical or non-critical for a process performed within the interior space.

15. The system according to claim 1, wherein the system is configured to record the interventions and parameters thereof, including at least one of date, time, duration and/or type of intervention time.

16. The system according to claim 1, wherein the system is configured to document the interventions.

17. The system according to claim 1, wherein the critical pharmaceutical operations comprise the production of medicine or medical nutrition.

18. The system according to claim 1, wherein the enclosure is equipped with instruments configured to produce medicine or medical nutrition.

19. The system according to claim 1, wherein the critical pharmaceutical operations comprise a pharmaceutical filling process, preferably an aseptic pharmaceutical filling process.

20. The system according to claim 1, wherein the interior space is an aseptic interior space.

21. The system according to claim 1, wherein the enclosure comprises at least one of a clean room class A, a glove box, an isolator, and a restricted access barrier system.

22. The system according to claim 1, wherein the enclosure comprises one or more glove ports.

23. The system according to claim 22, wherein the controller is further configured to determine whether or not a respective glove of each of the one or more glove ports is arranged inside or outside of a wall of the enclosure.

24. The system according to claim 22, wherein the pre-defined region of the respective image frames depicts at least one of the one or more glove ports.

25. A method for monitoring critical pharmaceutical operations, comprising:
receiving, by a controller, image frames recorded by at least one camera, the at least one camera being installed so as to record the image frames of an interior space defined by an enclosure,
analyzing, by the controller, the image frames using a first model to detect an event captured by one or more of the image frames,
performing, by the controller, a classification of an intervention captured by the one or more of the image frames using a second model, the second model being trained with image frames of interventions assigned to at least two different classes, and
providing, by the controller, a notification indicating one of the at least two different classes based on the classification.
